(19) United States
(12) Patent Application Publication
(10) Pub. No.: US 2017/0226042 A1
(43) Pub. Date: Aug. 10, 2017

(54) DEVICE METHOD OF MAKING ARTEPILLIN C IN PROPOLIS FOR ANTI-CANCER

(71) Applicant: Ultra-Microrigin Biomedical Technology Co., Ltd., Zhubei City (TW)

(72) Inventors: Ming-Hsi Chuang, Zhubei (TW); Chiu-Ying Peng, Zhubei (TW); Cheng-Yu Chi, Zhubei (TW); Hsin-Yu Chung, Zhubei (TW); Chu-Ting Liu, Zhubei (TW); Hui-Chen Kuo, Zhubei (TW)

(21) Appl. No.: 15/134,477

(22) Filed: Apr. 21, 2016

(30) Foreign Application Priority Data
Feb. 5, 2016 (TW) 105103946

(51) Int. Cl. C07C 51/47; C07C 59/52; G01N 33/50; C07C 51/42

(52) U.S. Cl. CPC C07C 51/47; C07C 51/42; C07C 59/52; G01N 33/5011

(57) ABSTRACT

A method of making artepillin C in propolis includes the following steps: A. Mix propolis with ethanol to obtain a propolis-ethanol extract; B. Provide supercritical carbon dioxide and the propolis-ethanol extract to a chromatographic column to separate wax and artepillin C, and then remove the wax at a bottom of the chromatographic column and collect the artepillin C containing isolate at a top of the chromatographic column; and C. Test the artepillin C by a cytotoxicity test, a cell morphology analysis, a cell cycle and apoptosis test, and a cell motility metastasis test to find an anti-cancer effect of the artepillin C containing isolate.

### BACKGROUND OF THE INVENTION

1. Technical Field

The present invention relates generally to active components of propolis, and more particularly to a method of making artepillin C from propolis for anti-cancer purpose.

2. Description of Related Art

Propolis has various physiological activities, such as antioxidant activity and anti-cancer activity. Many researches and papers have proven that propolis is effective in reducing the size of tumor, killing cancer cells, repressing growth and metastases of malignant tumor, reducing the side-effects of chemotherapy, increasing immunity, and reducing fibrosis. Such effects come from some components in propolis, including flavonols, artepillin C, enzyme, and organic acids, which may repress and kill cancer cells.

There is patent, such as China patent CN102885854B, disclosing that Taiwan green propolis extracts is helpful in increasing the effect of anti-cancer drugs, dilation of life, and reducing the size of tumor. However, green propolis extracts only work when it is taken in accompany with other anti-cancer medicines. Patent CN103285038A discloses a propolis composition for preventing and treating cancer and tumor; CN104095174A discloses that the purposes of extracting propolis is for anti-cancer, so it is important to have high quality and safety of propolis extracts; CN104523829A discloses a propolis recipe as a food supplement to anti-cancer, antitumor, and reduce side-effect of chemotherapy; and CN100536882C discloses a propolis containing Chinese medicine formula for treating cancer, increasing immunity, improving constitution, and increasing anti-cancer ability of patients. The last patent also mentions that it is effective to 92% treated patients.

European Patent, EP0976399B1, teaches a conventional method of extracting and purifying flavonoids and artepillin C from propolis. In this method, an organic solvent is used to remove wax in the propolis. But, the bad result is that flavonoids and artepillin C are damaged by the solvent, and residual organic solvent may raise health problem. Although there is known propolis extraction method by using supercritical CO₂ fluid, the method is not practical enough since it needs lots professional working experience and tedious testing and purification periods.

### BRIEF SUMMARY OF THE INVENTION

To improve the technique mentioned above, the primary objective of the present invention is to provide a method of making artepillin C from propolis that may produce high contraction artepillin C from a propolis-ethanol solution, and remove wax. Propolis is effective in anti-cancer, which has been proven in literatures. The method of the present invention is low cost, recyclable, safe, and practicable.

The present invention provides a method of making artepilin C in propolis including the following steps:

A. Mix propolis with ethanol to obtain a propolis-ethanol extract;

B. Provide supercritical carbon dioxide and the propolis-ethanol extract to a chromatographic column to separate wax and artepillin C to remove the wax at a bottom of the chromatographic column, and collect the artepillin C at a top of the chromatographic column, wherein a working pressure and a working temperature are set to 3,000-4,000 psi and 40-60° C., and a pressure and a temperature of the chromatographic column is set to 3,000-4,000 psi and 40-60° C., a flow rate of the supercritical carbon dioxide is set to 6-9 L/hr, and a flow rate of the propolis-ethanol extract is set to 1-3 L/hr; and

C. Test the artepillin C activity by test including cytotoxicity, cell morphology analysis, cell cycle and apoptosis test, and cell motility metastasis test to evaluate anti-cancer effect of the artepillin C.

Besides, the present invention also provides artepillin C, which is effective in anti-cancer, made by the aforesaid method.

### BRIEF DESCRIPTION OF THE SEVERAL VIEWS OF THE DRAWINGS

The present invention will be best understood by referring to the following detailed description of some illustrative embodiments in conjunction with the accompanying drawings, in which

FIG. 1 is a flow chart of a preferred embodiment of the present invention;

FIG. 2 is a diagram showing comparison of relative amount of wax remained after SFE extraction and conventional ethanol extract;

FIG. 3 is a comparative HPLC chromatogram mover laying the artepillin C content produced after SFE in red and conventional ethanol extract in black;

FIG. 4 is a diagram of the preferred embodiment of the present invention, showing the survival rates of HCT116 cells in the SFE propolis with different concentrations and time;

FIG. 5 is pictures depicting HCT116 morphological changes in the SFE propolis and that in control group;

FIG. 6 is a diagram of HCT116 distribution in cell cycle after SFE propolis treatment for 0, 24, and 48 hours; and

FIG. 7 is a diagram, showing the motility of HCT116 cells in the SFE propolis and that in control group after 0 hour and 24 hours.

### DETAILED DESCRIPTION OF THE INVENTION

The detailed description and technical contents of the present invention will be explained with reference to the accompanying drawings. However, the drawings are for illustration only and cannot be used to limit the present invention.

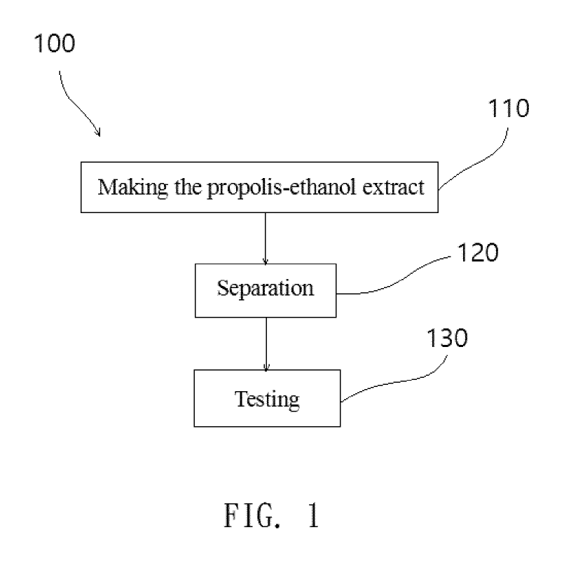

As shown in FIG. 1, a method 100 of making artepillin C containing isolate from propolis includes the following steps:

The first step is preparation of a propolis-ethanol extract solution 110, which includes adding propolis into ethanol until a saturation state is reached, then removing impurities, and then removing impurities again by a centrifugal separator to obtain the propolis-ethanol extract solution.

The second step is separation 120, which includes providing supercritical carbon dioxide and the propolis-ethanol extract solution to a chromatographic column to separate wax and artepillin C from the propolis-ethanol extract solution. Wax in the propolis-ethanol extract solution is accumulated on a bottom of the chromatographic column for removal, and the artepillin C containing isolate is collected at a top of the chromatographic column. A working pressure and a working temperature are set to 3,000-4,000 psi and 40-60° C., and a pressure and a temperature of the chromatographic column is set to 3,000-4,000 psi and 40-60° C., a flow rate of the supercritical carbon dioxide is set to 6-9 L/hr, and a flow rate of the propolis-ethanol extract is set to 1-3 L/hr.

The chromatographic column has a stainless container with 0.036-0.125 m in an interior diameter and 1 m in a height. A stainless plate is received in the container, and the plate is made of Pro-Pak protruded metal, saddles, rings, structured packing, or knitted packing.

The third step is testing 130, in which the artepillin C containing isolate is tested by cytotoxicity test, cell morphology analysis, cell cycle and apoptosis test, and cell motility metastasis test to find the anti-cancer effect of the artepillin C.

In the cytotoxicity test, HCT116 cells are put in a 96-well plate (10,000 cells/well), in which a 200 μL complete medium (McCoy's 5a) is received, and the complete medium is replaced with the artepillin C containing isolate (0.25-1.0 mg/mL) medium in the next day. The control group is added with 200 μL complete medium only. After 2 days, a ratio of a viable cell count in each well is tested by a (3-(4,5-dimethylthiazol-2-yl)-2,5-diphenyltetrazolium bromide (MTT) colorimetric assay, and an optical density in 570 nm is obtained by an ELISA reader.

In cell morphology analysis, HCT116 cells are received in a petri dish, and added with the artepillin C containing isolate (1.0 mg/mL) medium after the cells are attached to the bottom in the next day. The dish sits for predetermined times (2, 7, 14, 21, and 28 days), and then the cell morphology in the dish is observed and taken for pictures by an inverted microscope.

In cell cycle and apoptosis test, HCT116 cells are mixed with the artepillin C containing isolate (1.0 mg/mL) medium, then mixed with trypsin-EDTA, and then collected with a culture medium to obtain a solution. Next the solution is centrifuged to remove supernatant liquor thereof to obtain sediment. Next the sediment is washed by phosphate solution, and added with 1 mL 70% cool methanol, and then is kept in a 4° C. environment for a night. The solution is centrifuged again, then added 1 mL phosphate solution for suspension, then added 50 mg/mL propidiumiodide for 10 minutes photophobic effect, and then exacted by 532 nm beams. After that, HCT116 cells are tested in a 590±40 nm wavelength for testing fluorescence by flow cytometry.

In cell motility metastasis test, HCT116 cells are put in a 6-well plate (8,000 cells per well), and added with 2 mL complete medium (McCoy's 5a). In the next day, making a line by a 200 μL pipette tip, and replacing the complete medium with the artepillin C containing isolate (0.25-1.0 mg/mL) medium, and then taking a picture of the HCT116 cells to measure a width of Day 0 by a 100× microscope. The control group is replaced by 2 mL complete medium without any addition, and put for a day to take a picture for an opening closed status of Day 1. At last, a measurement software (Image J) is applied to measure widths between 5 points on each picture, and it may get at least 25 data for each condition to compare the averages of the widths of Day 0 and Day 1 that may get the cell motility ratio.

The following description is about the tests of wax and artepillin C, and the test of the effect of artepillin C of the present invention on anti-cancer.

In the test of wax, 5 C.C. SFE (Supercritical Fluid Extractor) propolis or purified propolisis added into 100C.C. water, and then the suspension is collected and dried to measure its weight that could obtain a content of wax in the SFE propolis. In the test of artepillin C, it is tested by a C18 chromatographic column and HPLC quantitative analyzer. A content of artepillin C is obtained by comparing with the standard curvature of a standard sample of artepillin C and an optical density of a quantitative sample under 320 nm wavelength.

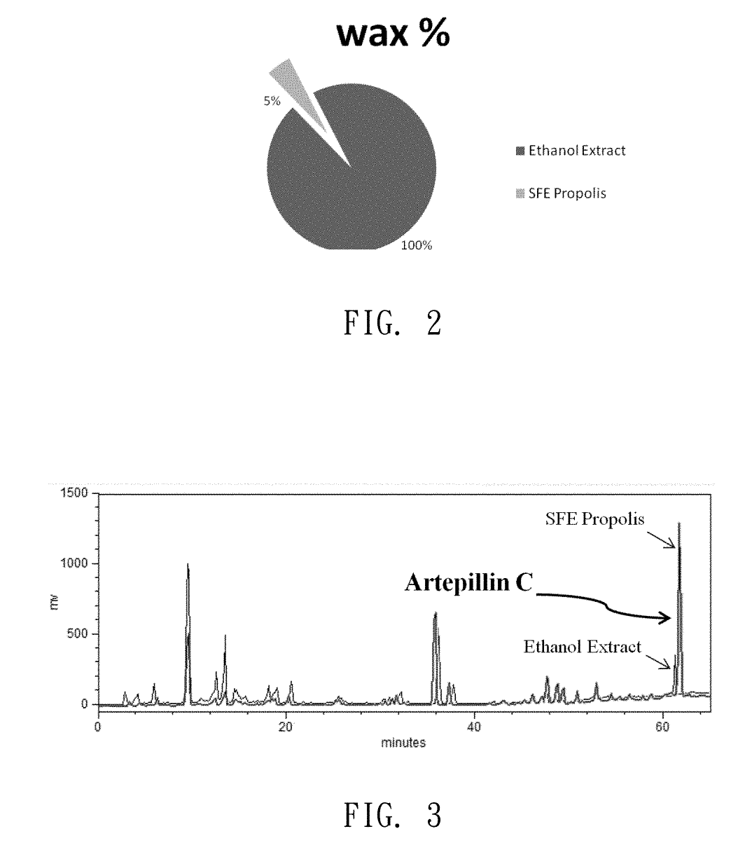

As shown in FIG. 2, in comparison with the SFE propolis of the present invention and the conventional ethanol extract propolis, there is 95% wax decreasing. It proves that the present invention is effective in removal of wax in propolis.

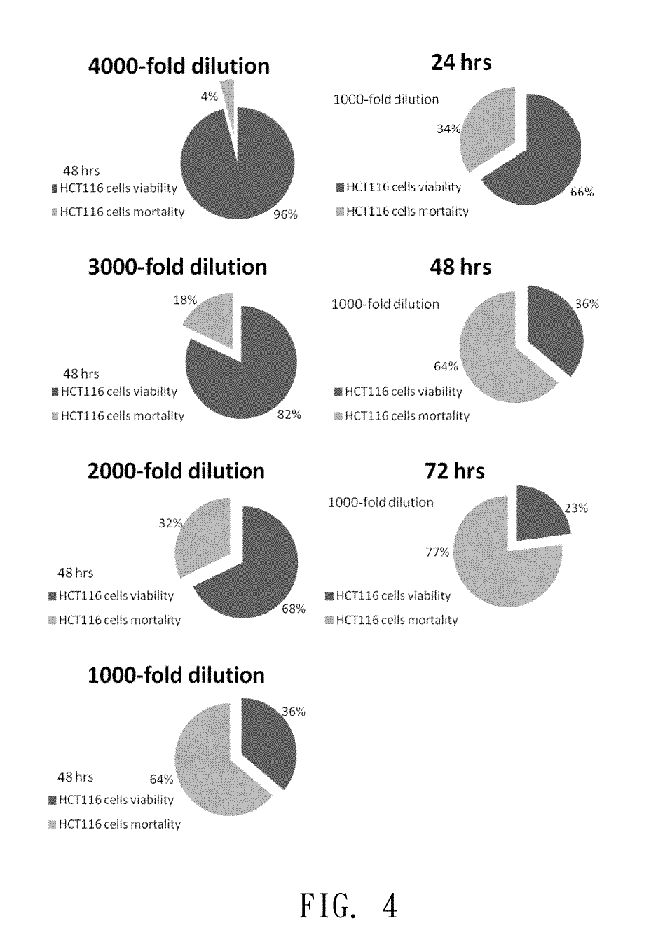

As shown in FIG. 3, artepillin C of propolis, which is the active anti-cancer component, is tested by HPLC, and it shows that the peaks of the artepillin C in the ethanol extract propolis are lower than that of the artepillin C in the SFE propolis of the present invention, which means that the SFE propolis of the present invention has more artepillin C.

In the test of anti-cancer cells, FIG. 4 (left) shows that HCT116 cells are added into SFE propolis with different concentrations. The results that the HCT116 cells viability increases while the concentration increases. It shows that the SFE propolis of the present invention is effective in killing cancer cells or repressing the growth of cancer cells. FIG. 4(left) further shows that 1,000-fold dilution is the most effective concentration, so the effectiveness of 1,000-fold dilution times is tested again for different treatment duration. As shown in right hand side of FIG. 4, the HCT116 cells viability decreases with longer treatment duration, which proves that the SFE propolis of the present invention is effective in killing cancer cells or repressing the growth of cancer cells when the patient takes it for a long time.

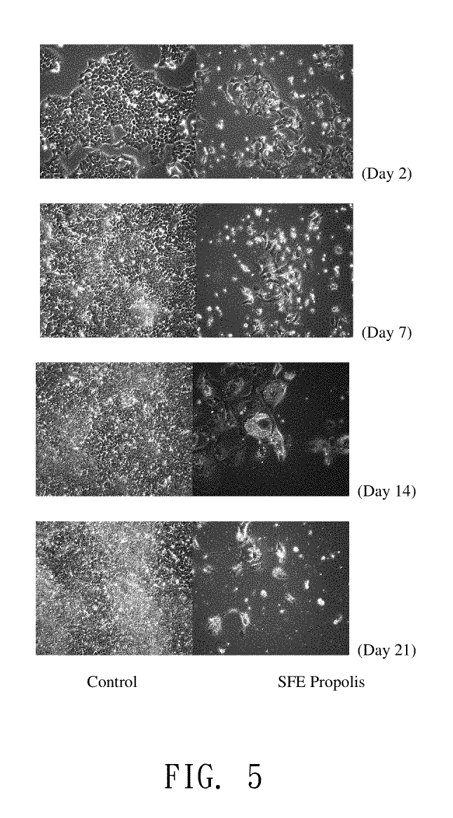

As shown in FIG. 5, for viability and cell morphology of SFE propolis of the present invention in comparison with the control group, viability and cell morphology are observed at the second, seventh, fourteenth, and twenty-first days. In regard to cytotoxicity, SFE propolis is more effective in repressing HCT116 cells than the control group. In regard to cell morphology, SFE propolis treated HCT116 cells change significantly, for example number of cells decreases and cells become rounder and larger. It shows that SFE propolis has the anti-cancer effect.

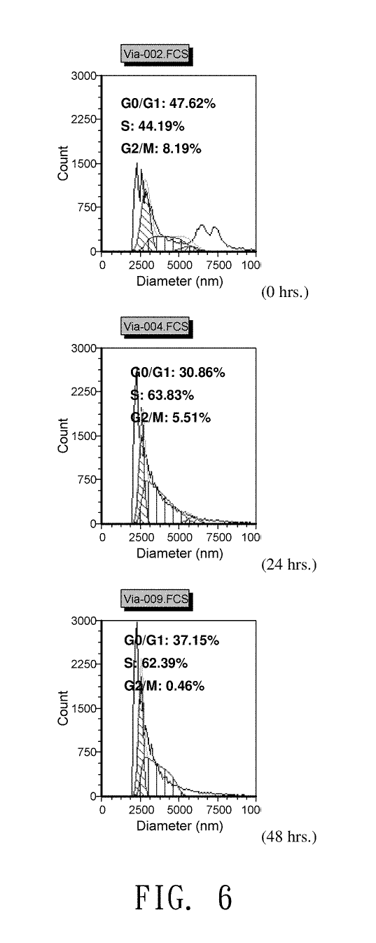

As shown in FIG. 6, cell cycle reflects the cell's status, cancer cells usually have abnormal division cycles that makes cancer cells grow faster than normal cells, so suppressing cell division in one of step of cell cycle is a potential way of anti-cancer. HCT116 cells are about equally distributed in G0/G1 and S phase. After SFE propolis treatment for 24 hours and 48 hours, a count of HCT116 cells at S stage increases, and a count of HCT116 cells at G0/G1 stage decreases, G2/M decreases as well. In comparison with the control group (0 hrs.), it has a significant difference in statistics (p<0.05). It is an evidence that SFE propolis is effective in repressing cancer cells to slow down or stop the growth of cancer cells.

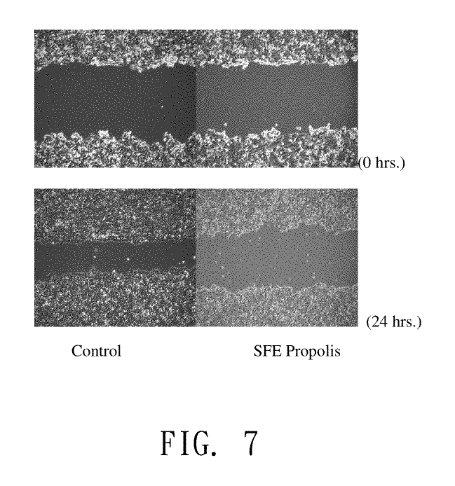

The test of wound healing is a simple and fast way to examine the effect on the cell motility (or metastasis), so as to examine tumorigenicity and metastasis of cancer cells. As shown in FIG. 7, the control group is unable to repress cancer cells, and the open wound is closing as time goes by. For SFE propolis treated HCT116 cells, the motility ability is repressed as the open wound is bigger than that of control group. The result in FIG. 7 shows that SFE propolis is significantly effective in repressing the growth of cancer cells, and the cell motility is less than that of the control group.

In conclusion, the method of the preferred embodiment of the present invention provides supercritical carbon dioxide as the solvent, and uses the chromatography and absorption techniques to massively separate and purify artepillin C. It could reach the balance with less ethanol and supercritical carbon dioxide under the supercritical condition. The present invention is effective in anti-cancer that has been proved by cytotoxicity test, cell morphology analysis, cell cycle and apoptosis test, and cell motility metastasis test. The method of the preferred embodiment of the present invention has a simple procedure, and no residual solvent problem. It further has some advantages, including low cost, recyclable, safety, and practicable.

It must be pointed out that the embodiments described above are only some preferred embodiments of the present invention. All equivalent structures which employ the concepts disclosed in this specification and the appended claims should fall within the scope of the present invention.

What is claimed is:

1. A method of making artepilin C in propolis, comprising the steps of:
A. adding propolis into ethanol until a saturation state is reached, and then removing impurities to obtain a propolis-ethanol extract;
B. providing supercritical carbon dioxide and the propolis-ethanol extract to a chromatographic column to separate wax and artepillin C from the propolis-ethanol extract, and then removing the wax at a bottom of the chromatographic column, and collecting the artepillin C at a top of the chromatographic column, wherein a working pressure and a working temperature are set to 3,000-4,000 psi and 40-60° C., and a pressure and a temperature of the chromatographic column is set to 3,000-4,000 psi and 40-60° C., a flow rate of the supercritical carbon dioxide is set to 6-9 L/hr, and a flow rate of the propolis-ethanol extract is set to 1-3 L/hr; and
C. testing the artepillin C by a cytotoxicity test, a cell morphology analysis, a cell cycle and apoptosis test, and a cell motility metastasis test to find an anti-cancer effect of the artepillin C containing isolate.

2. The method of claim 1, wherein the chromatographic column has a stainless container with 0.036-0.125 m in an interior diameter and 1 m in a height; a stainless plate is received in the container, which is made of a material selected from the group consisting of Pro-Pak protruded metal, saddles, rings, structured packing, and knitted packing.

3. The method of claim 1, further comprising a test of the wax in the step B, including adding 5 C.C. hepropolis-ethanol extract to 100 C.C. water; and collecting and drying a suspension dried to measure a weight thereof, so as to obtain a content of the wax in the propolis-ethanol extract.

4. The method of claim 1, further comprising a test of the artepillin C containing isolate in the step B, including providing a C18 chromatographic column to obtain a content of the artepillin C by comparing with a standard curvature of a standard sample and an optical density of a quantitative sample under 320 nm wavelength with a HPLC quantitative analyzer.

5. The method of claim 1, wherein the cytotoxicity test including providing HCT116 cells in a plate having 96 wells (10,000 cells/well), in which a 200 μL complete medium (McCoy's 5a) is received, and replacing the complete medium by a medium containing the SFE propolis (0.25-1.0 mg/mL) after a predetermined time, and adding the medium into other wells of the 96-well plate; adding a 200 μL complete medium only to be a control group, a ratio of a viable cell count in each of the well is tested by a (3-(4,5-dimethylthiazol-2-yl)-2,5-diphenyltetrazolium bromide (MTT) colorimetric assay, and an optical density in 570 nm is obtained by an ELISA reader.

6. The method of claim 1, wherein the cell morphology analysis includes providing HCT116 cells in a petri dish, and adding a medium containing the SFE propolis (10 mg/mL) in the petri dish and waiting for a time until the HCT116 cells are attached to a bottom of the petri dish, and then observing and taking a picture of the HCT116 cells in the petri dish by an inverted microscope.

7. The method of claim 1, wherein the cell cycle and apoptosis test includes mixing HCT116 cells with a medium containing the SFE propolis (10 mg/mL) and trypsin-EDTA, and then collecting with a culture solution to obtain a solution; centrifuging the solution, and then removing a supernatant liquor thereof to obtain a sediment; washing by a phosphate solution, and then adding 1 mL 70% cool methanol, and keeping in a 4° C. environment for a predetermined time; adding an 1 mL phosphate solution for suspension and 50 mg/mL propidiumiodide for a photophobic effect after centrifugation, and then exposing under 532 nm beams to test fluorescence of a 590±40 nm wavelength of the HCT 116 cells by flow cytometry.

8. The method of claim 1, wherein the cell motility metastasis test includes providing HCT116 cells in a plate having 6 wells (8,000 cells per well), and adding a 2 mL complete medium in the plate; making a line by a 200 μL pipette tip; replacing the complete medium by the SFE propolis (0.25-1.0 mg/mL) after a predetermined time, and then taking a picture of the HCT116 cells to measure a width of Day 0 by a 100× microscope; a control group having a 2 mL compete medium in a 6-well culture plate without any addition, and putting the 6-well culture plate for a day to take a picture of the HCT116 cells to measure an opening closed status of Day 1; measuring widths between 5 points on each of the pictures by a measurement software to obtain at least 25 data for each condition to compare averages of the widths of Day 0 and Day 1 that may get the cell motility ratio.

9. The method of claim 8, wherein the measurement software is Image J.

10. Artepillin C, which is effective in anti-cancer, made by the method as defined in claim 1.
